(19) United States
(12) Patent Application Publication
(10) Pub. No.: US 2026/0048181 A1
(43) Pub. Date: Feb. 19, 2026

(54) ANTIMICROBIAL POLYCARBONATE COATINGS

(71) Applicant: BOARD OF REGENTS, THE UNIVERSITY OF TEXAS SYSTEM, Austin, TX (US)

(72) Inventors: Joel ROSENBLATT, Pottstown, PA (US); Issam RAAD, Missouri City, TX (US)

(73) Assignee: BOARD OF REGENTS, THE UNIVERSITY OF TEXAS SYSTEM, Austin, TX (US)

(21) Appl. No.: 19/101,622

(22) PCT Filed: Aug. 22, 2023
(86) PCT No.: PCT/US2023/072620

§ 371 Date: Feb. 6, 2025

Related U.S. Application Data
(60) Provisional application No. 63/399,867, filed on Aug. 22, 2022.

(51) Int. Cl. A61L 31/10; A61L 31/16

(52) U.S. Cl. CPC A61L 31/10; A61L 31/16; A61L 2300/406

(57) ABSTRACT

Antimicrobial polycarbonate coatings are provided that may be included on the surface of a medical device such as, e.g., a needleless connector. The antimicrobial polycarbonate coatings may comprise minocycline, rifampin, and/or chlorhexidine. Methods of applying the antimicrobial polycarbonate coating to a medical device (e.g., a polycarbonate connector) are also provided.

This application claims the benefit of U.S. Provisional Patent Application No. 63/399,867, filed Aug. 22, 2022, the entirety of which is incorporated herein by reference.

### BACKGROUND OF THE INVENTION

### 1. Field of the Invention

The present invention relates generally to the field of molecular biology and medicine. More particularly, it concerns antimicrobial polycarbonate coatings that can be applied to or included on a medical device.

### 2. Description of Related Art

Microbial contamination of medical devices continues to be a problem in clinical environments such as hospitals. Vascular access devices represent a significant risk for microbial contamination. Although advances have been made in improving the antimicrobial properties of various medical devices such as catheters, including impregnating different antimicrobial compounds into the surface of a catheter, microbial colonization of medical devices continues to result in patient infections in hospitals and other medical settings.

Some vascular catheters fabricated from polyurethane have antimicrobial coatings. An advanced coating that provides durable broad-spectrum antimicrobial protection is described in U.S. Pat. No. 10,744,232. This patent discloses impregnation of minocycline and rifampin into a first surface of the aliphatic polyether polyurethane polymer catheter followed by sequential coating with an additional polymer layer containing chlorhexidine.

In many applications, it is desirable to have coatings that can reduce or prevent microbial colonization and/or thrombus formation on the devices. Clearly, there exists a need for broad spectrum antimicrobial coatings for polycarbonate devices.

### SUMMARY OF THE INVENTION

The present disclosure overcomes limitations in the prior art by providing antimicrobial polycarbonate coatings that may be included on a medical device such as, e.g., a needleless connector. The inventors tested applying antimicrobial aliphatic polyether polyurethane coatings of U.S. Pat. No. 10,744,232 to polycarbonate connectors, but the coating failed to bond the antimicrobial agents in a manner that did not immediately wash off the polycarbonate surface. Attempts to include the antimicrobial agents in polycarbonate during extrusion adversely affected the integrity or function of the antimicrobial agents and were not acceptable for use. Attempts to solution impregnate molded polycarbonate devices at temperatures that did not damage the antimicrobial agents, warp or otherwise change the device structure proved to be difficult or were not successful. The present disclosure is based, in part, on the development of solutions containing polycarbonate and one or more antimicrobial agents that can be applied to a medical device to form an antimicrobial polycarbonate coating. In some aspects, antimicrobial polycarbonate coatings are provided that effectively bind to the surface of a polymer (e.g., polycarbonate or polyurethane), are substantially transparent, and provide antimicrobial properties to polymer surface. The present disclosure is based, in part, on the discovery that blending a polycarbonate-solvent (e.g., dioxane or dioxalane) solution with a lower alcohol (e.g., methanol) solution containing an antimicrobial agent (e.g., minocycline, rifampin, and/or chlorhexidine diacetate) could be applied to a polycarbonate surface (e.g., via dipping, wiping, or spraying) to form a durable coating that was substantially transparent or translucent to allow for visualization of the flow of fluids, and exhibited broad antimicrobial properties. Use of polycarbonate solvent dioxane surprisingly resulted in more transparent coatings (which may advantageously be used for visualizing fluid flow through the device) than use of the polycarbonate solvents dioxalane or carbothane. The coating bonded strongly to the polycarbonate medical device, even when immersed in aqueous media and/or subjected to mechanical friction. The antimicrobial polycarbonate coating may change color over time as the antimicrobial agents elute from the surface coating. If desired different types or combinations of antimicrobial agents may be included in separate antimicrobial polycarbonate layers that are applied to the medical device. In some cases, a polycarbonate-only layer can be applied to the medical device prior to application of the antimicrobial polycarbonate coating. Spatial segregation of different antimicrobial polycarbonate layers may be separated or enhanced by application of a polycarbonate-only layer between the antimicrobial or bioactive layers. A polycarbonate-only topcoat can be applied, if desired, to further stabilize the bioactive agents or modulate their elution rates. The antimicrobial polycarbonate coating can be applied to a variety of medical devices. Methods of applying antimicrobial polycarbonate coatings are provided.

An aspect of the present disclosure relates to a medical device comprising an antimicrobial polycarbonate coating, wherein the antimicrobial coating comprises an antimicrobial agent, and polycarbonate; wherein the antimicrobial agent is minocycline, rifampin, or chlorhexidine; and wherein the antimicrobial coating and has been formed by applying a solution comprising the antimicrobial agent and the polycarbonate to a surface of the medical device and allowing the solution to dry. The solution may comprise dioxane, dioxalane, a chlorinated solvent, methylene chloride, chloroform, cresol, dimethylformamide, dimethylacetamide, or n-methyl pyrrolidone. In some embodiments, the solution comprises dioxane. The solution may comprise a C₁₋₄ alcohol (e.g., methanol). The polycarbonate may comprise the structure

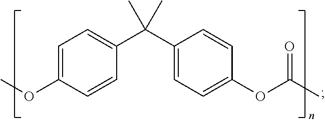

wherein n=4-18. The polycarbonate may comprise a clear polycarbonate, filled polycarbonate, polycarbonate blend, polycarbonate copolymer, polycarbonate block copolymer, high molecular weight polycarbonate, aliphatic polycarbonate, aromatic polycarbonate, or branched polycarbonate. In some embodiments, the polycarbonate comprises clear polycarbonate. In some embodiments, the ratio of polycarbonate to antimicrobial agent (w/w) is from about 2 to about 9, from about 2.25 to about 8, or from about 2.25 to about 4. The coating may comprise a second polymer that is blended with the polycarbonate. The second polymer may be a polyurethane, polypropylene, polyethylene, urethane, polyester, polyacrylate, acrylonitrile, rubber, polyalkenes, polysiloxane, polyethylene, polybutyl, polystyrene, polypropylene, polybutadiene, polysulfone, polyamide, or fluoropolymer. In some embodiments, the second polymer is a polyurethane. The polyurethane may comprise the structure

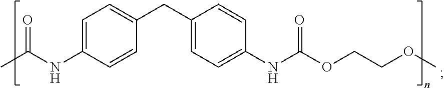

wherein n=4-18. The ratio of polycarbonate to the second polymer may be from about 99:1 (w/w) to about 50:50 (w/w), from about 95:5 (w/w) to about 65:45 (w/w), or from about 90:10 (w/w) to about 75:25 (w/w). In some embodiments, the antimicrobial polycarbonate coating comprises (i) minocycline and rifampin, (ii) minocycline and chlorhexidine, or (iii) rifampin and chlorhexidine. In some embodiments, the antimicrobial polycarbonate coating comprises minocycline and rifampin. The antimicrobial polycarbonate coating may comprise minocycline, rifampin, and chlorhexidine. In some embodiments, the chlorhexidine is present in the coating in an amount of less than about 54% (w/w), about 25-50% (w/w), or about 44% (w/w/) or less, when the coating is dried. The chlorhexidine may be chlorhexidine diacetate, or chlorhexidine dichloride, chlorhexidine dihydrochloride, chlorhexidine digluconate, or a chlorhexidine base. In some embodiments, the minocycline, rifampin, and/or chlorhexidine are dissolved in a C₁₋₄ alcohol to form a first mixture, and the polycarbonate is dissolved or dispersed in dioxane, dioxalane, a chlorinated solvent, methylene chloride, chloroform, cresol, dimethylformamide, dimethylacetamide, or n-methyl pyrrolidone to form a second mixture, and wherein the first mixture and the second mixture are combined to form the solution comprising the antimicrobial agent and the polycarbonate. The C₁₋₄ alcohol may be methanol. The polycarbonate may be dissolved or dispersed in dioxane. In some embodiments, the ratio of polycarbonate to dioxane is from about 60:40 to about 80:20, or from about 65:35 to about 75:25. In some embodiments, the minocycline, rifampin, and/or chlorhexidine are dissolved in methanol in an amount of about 0.1-2% (w/v) or about 0.5-1.5% (w/v). In some embodiments, the minocycline, rifampin, and/or chlorhexidine are dissolved in methanol with no or essentially no precipitation of the minocycline, rifampin, and/or chlorhexidine. The antimicrobial polycarbonate coating may further comprises an additional antimicrobial agent such as, e.g., an antibiotic, an antifungal agent, an antiseptic, an antimicrobial metal, a biofilm disrupter, a chelator, or a fatty acid. The chelator may be citrate, ethylene diaminedisuccinate (EDDS), tetrakis hydroxymethyl phosphonium sulfate (THPF), or EDTA. The fatty acid may be a C₆₋₁₂ alkanoic acid, more preferably a C₆₋₁₀ alkanoic acid. The fatty acid may be hexanoic acid, decanoic acid, dodecanoic acid, caprylic acid (octanoic acid), caproic acid, or lauric acid. In some embodiments, the fatty acid is caprylic acid (octanoic acid). The antibiotic may be a glycopeptide, a macrolide, a betalactam, a quinolone, a cephalasporin, a tetracycline, an aminoglycoside, a sulfa drug, an oxazolidinone, a nitrofuran, a betalactamase inhibitor, trimethoprim, tobramycin, clindamycin, doxycycline, tigecycline, or a combination thereof. The antifungal agent may be a polyene, an azole, an echinocandin, a mycovirus, or a mycophage. The antiseptic may be a peroxide, an antimicrobial metal compounds (e.g., silver, copper, zinc, titanium, barium, gold, gallium, nickel, aluminum, tellurium, selenium), a halogen (e.g., chlorine, iodine), sulfur, a sulfur compound, a phenolic, a quaternary ammonium compound, a halogenated phenol, an antimicrobial dye (e.g., methylene blue, brilliant green, crystal violet), an anatase titanium dioxide, a guanidium compound, a nitric oxide donor (e.g., SNAP and GSNO), a thionitrite, a NONOate, an antimicrobial peptide, an antimicrobial lipid (e.g., a fatty acid), a bacteriophage, a polymer comprising any of the foregoing, a nanoparticle (e.g., a nanoemulsion or nanosuspension) comprising any of the foregoing, or a combination thereof. The antimicrobial polycarbonate coating may further comprise an additional therapeutic agent. The additional therapeutic agent may be an anticoagulant, a platelet inhibitor, a direct thrombin inhibitor, a calcium channel blocker, inhibitors of platelet or thrombin activation, thrombolytic agent, or a vasodialator (e.g., glyceryl trinitrate). In some embodiments, the additional therapeutic agent is an argatroban, a dabigatran, lepirudin, or bivalirudin. The antimicrobial polycarbonate coating may comprise nanoparticles (e.g., a nanoemulsion or nanosuspension). The nanoparticles may comprise gold or a therapeutic agent, e.g., as described herein or above. The thickness of the antimicrobial polymeric coating may be from about 1 micron to about 1 mm. The applying may comprise dip-coating, painting, wiping, or spray coating. The antimicrobial polycarbonate coating may be translucent or substantially transparent. In some embodiments, the antimicrobial polycarbonate coating includes a visual cue to indicate that antimicrobial protection is depleting or wherein the color of the polycarbonate can change over time due to depletion of the antimicrobial agent from the antimicrobial polycarbonate coating. In some embodiments, the medical device is a connector, and wherein the color of the polymeric coating can fade or become clearer due to elution of the minocycline and/or rifampin. In some embodiments, the medical device is a needleless connector, and wherein the needleless connector comprises a silicone seal and an internal cannula. The needleless connector may comprise a split septum, a straight fluid path, and a clear or substantially transparent housing. The surface of the medical device may comprise polycarbonate, polyvinyl chloride (PVC), polypropylene (PP), polyethylene (PE), and polystyrene (PS), polyurethane, polysiloxane, fluoropolymer, polyester, or polyamide. In some embodiments, the surface of the medical device comprises or consists of polycarbonate. In some embodiments, the medical device is a needleless connector, wherein the needleless connector comprises a polycarbonate housing. The antimicrobial polycarbonate coating may comprises multiple polycarbonate layers. The multiple layers may comprise at least 1, 2, or 3 layers that comprise the minocycline, rifampin, and/or chlorhexidine. The antimicrobial polycarbonate coating may comprise at least 2 layers that that comprise the minocycline, rifampin, and/or chlorhexidine that are separated by a polycarbonate layer that contains no or essentially no antimicrobial agent. In some embodiments, the medical device comprises a first polycarbonate layer and a second polycarbonate layer; wherein the first polycarbonate layer comprises minocycline and rifampin, and wherein the second polycarbonate layer comprises chlorhexidine. The first polycarbonate layer and the second polycarbonate layer may be separated by a third polycarbonate layer, wherein the third polycarbonate layer comprises no or essentially no antimicrobial agent. The antimicrobial polycarbonate coating may comprise a polycarbonate layer that comprises minocycline, rifampin, and chlorhexidine. In some embodiments, the antimicrobial polycarbonate coating comprises a polycarbonate topcoat, wherein the polycarbonate topcoat comprises no or essentially no antimicrobial agent. In some embodiments, the medical device comprises a polycarbonate layer comprises no or essentially no antimicrobial agent that is below the polycarbonate layers that contain the antimicrobial agent. In some embodiments, the medical device is a connector, clip, staple, housing, cartridge, trocar, tube, barrel, plunger, or inflator. In some embodiments, the medical device is a connector such as, e.g., a needleless connector.

Another aspect of the present disclosure relates to a kit comprising the medical device as described above or herein and a container means. The medical device may be a connector such as, e.g., a needleless connector.

Yet another aspect of the present disclosure relates to a method of reducing infection in a mammalian subject comprising using the medical device as described above or herein in a medical procedure on the mammalian subject. The medical device may be a connector, clip, staple, housing, cartridge, trocar, tube, barrel, plunger, or inflator. In some embodiments, the medical device is a connector and wherein the medical procedure is intravenously administering a fluid to the mammalian subject via the connector. In some embodiments, the connector comprises minocycline and/or rifampin, and wherein the becomes clearer or less colored over time as the fluid passes through the connector. The connector may be a needleless connector.

Another aspect of the present disclosure relates to a method of producing an antimicrobial polycarbonate coating, comprising: (i) dissolving or dispersing an antimicrobial agent in a C₁₋₄ alcohol, to form a first solution; (ii) dissolving or dispersing a polycarbonate in a solvent to form a second solution; (iii) mixing the first solution and the second solution to form a third solution; (iv) applying the third solution to a surface; and (v) substantially drying the third solution to form the antimicrobial polycarbonate coating; wherein the antimicrobial agent is minocycline, rifampin, or chlorhexidine. The surface may be a on a medical device. In some embodiments, the surface is a polycarbonate surface on a medical device. The applying may comprise dip-coating, painting, wiping, or spray coating. The drying may occur at room temperature. The drying may comprise the application of heated air or dry air. The solvent may comprise dioxane, dioxalane, a chlorinated solvent, methylene chloride, chloroform, cresol, dimethylformamide, dimethylacetamide, or n-methyl pyrrolidone. In some embodiments, the solvent comprises dioxane. The C₁₋₄ alcohol may be methanol. The polycarbonate may comprises the structure

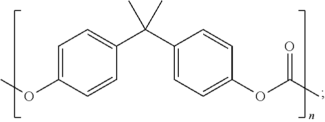

wherein n=4-18.

The polycarbonate may comprise a urethane, polyester, polyacrylate, acrylonitrile, rubber, polyalkene, polysiloxane, polyethylene, polybutyl, polystyrene, polypropylene, polybutadiene, polysulfone, polyamide, or fluoropolymer. In some embodiments, the polycarbonate comprises clear polycarbonate. In some embodiments, the ratio of polycarbonate to antimicrobial agent (w/w) in the dried antimicrobial polycarbonate coating is from about 2 to about 9, from about 2.25 to about 8, or from about 2.25 to about 4. In some embodiments, the C₁₋₄ alcohol is methanol; and wherein the solvent is dioxane. The ratio of polycarbonate to dioxane in the second solution may be from about 60:40 to about 80:20 or from about 65:35 to about 75:25. In some embodiments, the first solution comprises minocycline, rifampin, and/or chlorhexidine in methanol in an amount of about 0.1-2% (w/v) or about 0.5-1.5% (w/v). In some embodiments, the first solution comprises minocycline, rifampin, and/or chlorhexidine dissolved in methanol with no or essentially no precipitation of the minocycline, rifampin, and/or chlorhexidine. The second solution may comprises a second polymer that is blended with the polycarbonate. The second polymer may be a polyurethane, polypropylene, polyethylene, urethane, polyester, polyacrylate, acrylonitrile, rubber, polyalkene, polysiloxane, polyethylene, polybutyl, polystyrene, polypropylene, polybutadiene, polysulfone, polyamide, or fluoropolymer. In some embodiments, the second polymer is a polyurethane. The polyurethane may comprise the structure

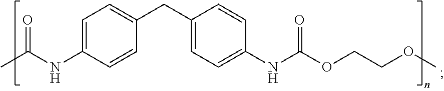

wherein n=4-18. In some embodiments, the ratio of polycarbonate to the second polymer in the dried antimicrobial polycarbonate coating is from about 99:1 (w/w) to about 50:50 (w/w), from about 95:5 (w/w) to about 65:45 (w/w), or from about 90:10 (w/w) to about 75:25 (w/w). The first solution may comprises (i) minocycline and rifampin, (ii) minocycline and chlorhexidine, or (iii) rifampin and chlorhexidine. In some embodiments, the first solution comprises minocycline and rifampin. The first solution may comprise minocycline, rifampin, and chlorhexidine. In some embodiments, the first solution comprises chlorhexidine in methanol in an amount of less than about 3.6% (w/v), or about 2.4% (w/v) or less. The chlorhexidine may be chlorhexidine diacetate, or chlorhexidine digluconate, chlorhexidine dichloride, or chlorhexidine dihydrochloride. The third solution may further comprise an additional antimicrobial agent. The additional antimicrobial agent may be an antibiotic, an antifungal agent, an antiseptic, an antimicrobial metal, a biofilm disrupter, a chelator, or a fatty acid. The chelator may be citrate, ethylene diaminedisuccinate (EDDS), tetrakis hydroxymethyl phosphonium sulfate (THPF), or EDTA. The fatty acid may be a C₆₋₁₂ alkanoic acid, more preferably a C₆₋₁₀ alkanoic acid. The fatty acid may be hexanoic acid, decanoic acid, dodecanoic acid, caprylic acid (octanoic acid), caproic acid, or lauric acid. In some embodiments, the fatty acid is caprylic acid (octanoic acid). The antibiotic may be a glycopeptide, a macrolide, a betalactam, a quinolone, a cephalasporin, a tetracycline, an aminoglycoside, a sulfa drug, an oxazolidinone, a nitrofuran, a betalactamase inhibitor, trimethoprim, tobramycin, clindamycin, doxycycline, tigecycline, or a combination thereof. The antifungal agent may be a polyene, an azole, an echinocandin, a mycovirus, or a mycophage. The antiseptic may be a peroxide, an antimicrobial metal compounds (e.g., silver, copper, zinc, titanium, barium, gold, gallium, nickel, aluminum, tellurium, selenium), a halogens (e.g., chlorine, iodine), sulfur, a sulfur compound, a phenolic, a quaternary ammonium compound, a halogenated phenol, an antimicrobial dye (e.g., methylene blue, brilliant green, crystal violet), an anatase titanium dioxide, a guanidium compound, a nitric oxide donor (e.g., SNAP and GSNO), a thionitrite, a NONOate, an antimicrobial peptide, an antimicrobial lipid (e.g., a fatty acid), a bacteriophage, a polymer comprising any of the foregoing, a nanoparticle (e.g., a nanoemulsion or nanosuspension) comprising any of the foregoing, or a combination thereof. The third solution may further comprise an additional therapeutic agent. The additional therapeutic agent may be an anticoagulant, a platelet inhibitor, a direct thrombin inhibitor, a calcium channel blocker, inhibitors of platelet or thrombin activation, thrombolytic agent, or a vasodialator (e.g., glyceryl trinitrate). In some embodiments, the additional therapeutic agent is an argatroban, a dabigatrans, lepirudin, or bivalirudin. The third solution may comprise nanoparticles (e.g., a nanoemulsion or nanosuspension). The nanoparticles may comprise gold or a therapeutic agent (e.g., as described above or herein). The antimicrobial polycarbonate coating may be translucent or substantially transparent. In some embodiments, the antimicrobial polycarbonate coating comprises minocycline and/or rifampin, and wherein the color of the polymeric coating can fade or become clearer with use. In some embodiments, the surface is on a medical device, wherein the medical device is a connector, and wherein the color of the polymeric coating can fade or become clearer due to elution of the minocycline and/or rifampin. In some embodiments, the surface is on a needleless connector, and wherein the needleless connector comprises a silicone seal and an internal cannula. The needleless connector may comprise a split septum, a straight fluid path, and a clear or substantially transparent housing. The surface may comprise polycarbonate, polyvinyl chloride (PVC), polypropylene (PP), polyethylene (PE), and polystyrene (PS), polyurethane, polysiloxane, fluoropolymer, polyester, or polyamide. In some embodiments, the surface comprises or consists of polycarbonate. In some embodiments, the medical device is a needleless connector, wherein the needleless connector comprises a polycarbonate housing. The method may comprise repeating steps (i)-(v), or wherein the method further comprises applying a fourth solution comprising the polycarbonate and the solvent to the surface. The method may comprise repeating steps (i)-(v), to produce at least 1, 2, or 3 layers in the antimicrobial polycarbonate coating that comprise minocycline, rifampin, and/or chlorhexidine. The method may comprise applying a fourth solution comprising the polycarbonate and the solvent to the surface, in order to produce at least two layers that that comprise the minocycline, rifampin, and/or chlorhexidine that are separated by a polycarbonate layer that contains no or essentially no antimicrobial agent. In some embodiments, the dried antimicrobial polycarbonate coating comprises a first polycarbonate layer and a second polycarbonate layer; where the first polycarbonate layer comprises minocycline and rifampin, and wherein the second polycarbonate layer comprises chlorhexidine. In some embodiments, the first polycarbonate layer and the second polycarbonate layer are separated by a third polycarbonate layer, wherein the third polycarbonate layer comprises no or essentially no antimicrobial agent. The dried antimicrobial polycarbonate coating may comprise a polycarbonate layer that comprises minocycline, rifampin, and chlorhexidine. The method may further comprise applying a polycarbonate topcoat, wherein the polycarbonate topcoat comprises no or essentially no antimicrobial agent. The method may further comprises applying a polycarbonate basecoat, wherein the polycarbonate basecoat comprises no or essentially no antimicrobial agent. In some embodiments, the surface is on a medical device, wherein the medical device is a connector, clip, staple, housing, cartridge, trocar, tube, barrel, plunger, or inflator. The medical device may be a connector such as, e.g., a needleless connector. When multiple polymer layers are applied to the surface, it is anticipated that the layers may be applied substantially one after another in a single process, or a period of time may be allowed to pass (e.g., 1-60 minutes, 1-24 hours, 1-2 weeks or more) between application of the different layers; for example, if desired, a layer can be applied to the surface and allowed to dry (e.g., via the application of heated or dried air) prior to application of the next layer(s) of the antimicrobial polycarbonate coating.

The type of nosocomial infection that can be reduced or prevented in various embodiments include, but are not limited to, pneumonia, bacteremia, fungimia, candidemia, a urinary tract infection, a catheter-exit site infection, and a surgical wound infection. Nosocomial infections that can be reduced or substantially prevented may be caused by bacteria such as, e.g., drug resistant bacteria. Some non-limiting example of drug resistant bacteria include methicillin-resistant staphylococcus, vancomycin-resistant enterococcus, and resistant Pseudomonas aeruginosa. The nosocomial infection may be caused by a fungus such as, e.g., a drug resistant fungi. Examples of a drug resistant fungi include members of the Candida genus. Infection by other pathogenic organisms that can cause the nosocomial infections may be reduced or prevented by use of the methods and medical devices, such as catheters, as described herein.

In various aspects, the antimicrobial agents may reduce the growth of a wide variety of bacterial and fungal organisms. The bacteria may be spherical, rod-shaped, or spiral bacteria. Non-limiting examples of bacteria include staphylococci (e.g., Staphylococcus epidermidis, Staphylococcus aureus), Enterococcus faecalis, Pseudomonas aeruginosa, Escherichia coli, among other gram-positive bacteria and gram-negative bacilli. Non-limiting examples of fungal organisms include Candida albicans and Candida krusei.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the inherent variation in the method being employed to determine the value, the variation that exists among the study subjects, or a value that is within 10% of a stated value.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The terms “subject,” “host,” “patient,” and “individual” are used interchangeably herein to refer to any mammalian subject for whom therapy is desired, particularly humans. Other subjects may include cattle, dogs, cats, guinea pigs, rabbits, rats, mice, horses, and so on.

The term “effective amount” is an amount sufficient to effect beneficial or desired clinical results. An effective amount can be administered in one or more administrations. For purposes of this application, an effective amount is an amount that is sufficient to palliate, ameliorate, stabilize, reverse, slow or delay the progression of the disease state. This includes, but is not limited to, a reduction in the frequency or severity of the signs or symptoms of a disease. For example, treatment of cancer may involve, for example, a reduction in the invasiveness of a tumor, reduction in the growth rate of the cancer, or prevention of metastasis. Treatment of cancer may also refer to prolonging survival of a subject with cancer.

An effective response of a patient or a patient's “responsiveness” to treatment refers to the clinical or therapeutic benefit imparted to a patient at risk for, or suffering from, a disease or disorder. Such benefit may include cellular or biological responses, a complete response, a partial response, a stable disease (without progression or relapse), or a response with a later relapse. For example, an effective response can be reduced tumor size or progression-free survival in a patient diagnosed with cancer.

“Treatment” and “treating” refer to administration or application of a therapeutic agent to a subject or performance of a procedure or modality on a subject for the purpose of obtaining a therapeutic benefit of a disease or health-related condition.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Photograph of a triple layer coated connector.

FIG. 2: Photograph of a topcoated antimicrobial connector.

### DESCRIPTION OF ILLUSTRATIVE EMBODIMENTS

### I. Antimicrobial Polycarbonate Coatings

In some aspects, antimicrobial polycarbonate coatings are provided that may be applied to or included on the surface of a medical device. These polymeric coatings may cover just a portion of or the entire device. If the polymeric coating only covers a portion of the device, then the portion covered by may be a defined segment or component of the device, a single surface, or part of a surface of the medical device. The portion of the device that is covered may the entire segment or the entire component. Medical devices provided here that comprise the antimicrobial polycarbonate coating may be made of a variety or polymers such as, e.g., polycarbonate or polyurethane. The antimicrobial polycarbonate coating may include one or more antimicrobial agents (e.g., minocycline, rifampin, and/or chlorhexidine), optionally in combination with one or more additional therapeutic compounds (e.g., anticoagulant, antithrombotic agent, etc.). In some embodiments, the antimicrobial polycarbonate polymer contains polycarbonate, optionally blended with a second polymer (e.g., a polyurethane carbamate) and/or a copolymer. The polymeric coatings provided herein preferably comprise a thermostable polycarbonate, and the polycarbonate can be optionally blended with one or more additional polymers such as a polyurethane polymer or other copolymer. In some preferred embodiments, the antimicrobial polycarbonate coating is substantially transparent or translucent.

Polycarbonate polymers are polymers comprising one or more repeating units that have a carbonate group. A polycarbonate polymer comprises one or more repeating units linked with a —OC(O)O— group. The polycarbonate polymers may further comprise an aliphatic or aromatic linking group between one or more of the —OC(O)O— groups. These linking groups may comprise or be joined by a heteroatom linker group such as an oxygen, sulfur, or nitrogen atom. These linking groups may comprise from about 2 to about 30 carbon atoms in each repeating unit, ideally from about 4 to about 18 carbon atoms. A model polymer is shown below.

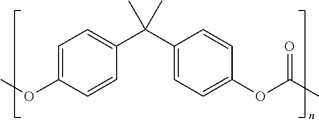

Polycarbonate polymers can provides advantages of increased light transmission and physical toughness. Polycarbonate polymers are typically highly transparent with light transmission often approaching that of glass, superior rigidity particularly under physiological conditions, shape retention, toughness and can withstand sterilization procedures. Polycarbonate polymers are particularly useful in medical devices for this reason. In some embodiments, an antimicrobial polycarbonate coating provided herein is applied to or included on a medical device made of polycarbonate (e.g., a polycarbonate needleless connector).

It is anticipated that a variety of polycarbonates can be included in the antimicrobial polycarbonate coating. In some embodiments, the polycarbonate coating includes a single polycarbonate. In other embodiments, the polycarbonate can include two or more different polycarbonates blended together to form the polycarbonate portion of the antimicrobial polycarbonate coating. Polycarbonates that can be included in an antimicrobial polycarbonate coating as described herein include, but are not limited to, clear polycarbonate, filled polycarbonates, polycarbonate blends, polycarbonate copolymers, polycarbonate block copolymers, high molecular weight polycarbonates, aliphatic polycarbonates, aromatic polycarbonates, branched polycarbonates,

The wt. ratio of polycarbonate (e.g., the polycarbonate alone, or the polycarbonate blend) to antimicrobial agent (w/w) in the coating may be about 2, 2.25, 2.4, 2.5, 3, 3.5, 3.9, 4, 4.5, 5, 6, 6.5, 7.1, 7.2, 7.3, 7.4, 7.5, 7.6, 7.7, 7.8, 7.9, 8, 8.25, 8.5, 9 or any range derivable therein, (e.g., about 3.9 or 7.8). These methods can be used to produce antimicrobial polycarbonate coatings that are substantially transparent or translucent.

In some embodiments, the antimicrobial polycarbonate coating is applied to the surface of a medical device (e.g., a needleless connector), wherein the antimicrobial polycarbonate coating and the surface comprise or are made of the same polycarbonate. In some embodiments, the antimicrobial polycarbonate coating is applied to the surface of a medical device (e.g., a needleless connector), wherein the antimicrobial polycarbonate coating and the surface comprise or are made of different polycarbonates. In some embodiments, the antimicrobial polycarbonate coating is applied to the surface of a medical device that is made of or comprises a polymer that is not a polycarbonate (e.g., wherein the surface of the medical device is a polyurethane, polypropylene, polyethylene, siloxane, vinyl, acrylate, glass, or metal).

### A. Polycarbonate Blends

The antimicrobial polycarbonate may comprise a blend of polymers that comprises a polycarbonate in combination with a second polymer (e.g., a polyurethane) and/or a copolymer. It is anticipated that a variety of polymers can be mixed with the polycarbonate at a ratio up to about 50:50 (w/w). In some embodiments, the ratio or polycarbonate to other polymers (e.g., the second polymer and/or copolymer) in the polycarbonate blend is about 99:1, 95:5, 90:10, 85:15, 80:20, 75:25, 70:30, 65:35, 60:40, 55:45, 51:49, or 50:50 (w/w). The antimicrobial polycarbonate coating may comprise a polycarbonate blend.

The second polymer may be a polyurethane polymer such as, e.g., a polyurethane polymer comprising one or more repeating units that include a carbamate group. A polyurethane polymer comprises one or more repeating units linked with a —OC(O)NR— or —NRC(O)O— group. The polyurethane polymers may further comprise an aliphatic or aromatic linking group between the one or more of the —OC(O)NR— or —NRC(O)O— groups. These linking groups may comprise or be joined by a heteroatom linker group such as an oxygen, sulfur or nitrogen atom. These linking groups may comprise from about 2 to about 30 carbon atoms in each repeating unit, ideally from about 4 to about 18 carbon atoms. A model polymer is shown below.

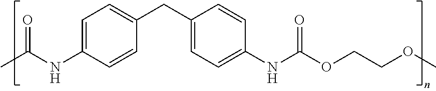

It is anticipated that a variety of different polymers can be blended with the polycarbonate without adversely affecting the properties of the resulting antimicrobial polycarbonate coating. In some embodiments, the second polymer is a urethane, polyester, polyacrylate, acrylonitrile, rubber, polyalkene, polysiloxane, polyethylene, polybutyl, polystyrene, polypropylene, polybutadiene, polysulfone, polyamide, or fluoropolymer.

In some embodiments, the antimicrobial polycarbonate coating comprises a copolymer. A copolymer is a polymer comprising two or more structurally distinct repeating units. These structurally distinct repeating units may be of the same type of polymer or a different type of polymer. For example, the polymer may comprise carbonate repeating units and urethane repeating units. The polymers may also comprise repeating units that are neither a polyurethane or a polycarbonate such as a propylene glycol or an ethylene glycol repeating unit.

Copolymers that can be blended with the polycarbonate include, e.g., urethanes, polyesters, polyacrylates, acrylonitriles, rubbers, polyalkenes, polysiloxanes, polyethylenes, polybutyls, polystyrenes, polypropylenes, polybutadienes, polysulfones, polyamides, and fluoropolymers.

Translucent polycarbonate blend coating layers are provided herein and can bonded to the surface of a medical device such as a polycarbonate device. As shown in the below examples, polycarbonate-solvent (e.g., dioxane) solutions can be blended with polyurethane-THF solutions in up to a 50:50 ratio (w/w), and the miscibility of the solutions can be maintained for a sufficient time to coat a polycarbonate device (e.g., a needleless connector). One or more bioactive agents (e.g., antimicrobial agents) may be included in a methanol solution that is added to the polycarbonate-solvent (e.g., dioxane) blended solution prior to application to the device, in order to produce an antimicrobial polycarbonate coating that contains a polycarbonate blend. The polymer blend solutions with bioactives can be processed in manners analogous to non-blended polycarbonate solutions described herein.

### B. Layering of Polycarbonate Coatings

In some embodiments, the antimicrobial polycarbonate coating comprises multiple layers. The layers may contain different antimicrobial agents. In some embodiments, the layers of the polycarbonate coating that contain one or more antimicrobial agents are separated by a polycarbonate layer that does not comprise an antimicrobial agent. In some embodiments, the outer layer of the polycarbonate coating does not include an antimicrobial agent when it is applied to the surface of a medical device, and it is anticipated that including a polycarbonate layer that does not contain an antimicrobial agent on the outer surface of the antimicrobial polycarbonate coating may, in some instances, improve the physical toughness or handling properties of the medical device comprising the antimicrobial polycarbonate coating.

The medical devices described herein may comprise using multiple layers of polymer coatings comprising polycarbonate to improve the performance of the composition. For example, the device may comprise a layer of the polymer only, then a polymer layer that comprises a functional molecule such as an antimicrobial agent and/or additional therapeutic agent. For example, in a layered antimicrobial polycarbonate coating, different layers may contain any of: minocycline, rifampin, chlorhexidine, (minocycline and rifampin), (minocycline and chlorhexidine), (rifampin and chlorhexidine), (minocycline, rifampin, and chlorhexidine), or no antimicrobial agent; and each of the foregoing may optionally include an additional therapeutic agent as described herein.

It is anticipated that a variety of additional therapeutic agents can be included in an antimicrobial polycarbonate coating. The functional molecule embedded in the polymer coating may be a biologically active molecule or active pharmaceutical ingredient (API) such as, e.g., an antibiotic, a blood thinner, a diuretic, an anticoagulant, an antithrombotic agent, an API that reduces clotting, a biologically active molecule such as a nucleic acid, a peptide, a protein, a sugar or polysaccharide, or a lipid. The device may also be coated with another polymer or polycarbonate layer after or on top of the layer containing the therapeutic agent or functional molecule has been attached to the medical device. This particular layer may be referred to as a top coat.

### C. Antimicrobial Agents

The antimicrobial polycarbonate coatings provided herein may include a variety of antimicrobial agents. In some preferred embodiments, the antimicrobial polycarbonate coating comprises minocycline, rifampin, and/or chlorhexidine. In some embodiments, the coating comprises minocycline and rifampin. As shown in the below examples, it has been surprisingly observed that antimicrobial polycarbonate coatings that contain reduced amounts of chlorhexidine (e.g., less than 3.6% or about 1.5-3% chlorhexidine diacetate in methanol for a coating solution) can exhibit superior microbial killing properties as compared to corresponding antimicrobial polycarbonate coatings that contain higher amounts or concentrations of chlorhexidine. The antimicrobial agent(s) in the antimicrobial polycarbonate coating may confer resistance to a variety of bacteria or microbes such as, e.g., Staphylococcus aureus, Pseudomonas aeruginosa, and/or Candida albicans.

Rifampin is a bactericidal antibiotic drug of the rifamycin group. The IUPAC systematic name for rifampin is (7S,9E,11S,12R,13S,14R,15R,16R,17S,18S,19E,21Z)-2,15,17,27,29-pentahydroxy-11-methoxy-3,7,12,14,16,18,22-heptamethyl-26-{(E)-[(4-methylpiperazin-1-yl)imino]methyl}-6,23-dioxo-8,30-dioxa-24-azatetracyclo[23.3.1.14,7.05,28]triaconta-1(28),2,4,9,19,21,25(29),26-octaen-13-yl acetate. Minocycline may also be incorporated into the polyether polyurethane surface or body of the device. Minocycline is a broad spectrum tetracycline based antibiotic with an IUPAC systematic name of (2E,4S,4aR,5aS,12aR)-2-(amino-hydroxy-methylidene)-4,7-bis(dimethylamino)-10,11,12a-trihydroxy-4a,5,5a,6-tetrahydro-4H-tetracene-1,3,12-trione.

Chlorhexidine (N′,N′″″-hexane-1,6-diylbis[N-(4-chlorophenyl)(imidodicarbonimidic diamide)]) is a small molecule antiseptic which can be effective against Gram-positive as well as Gram-negative bacteria. In some embodiments, chlorhexidine may be used in combination with, or may be substituted with, another antimicrobial guanidium compound such as, e.g., alexidine, hexamidine, polyhexamethylbiguanide or a chlorhexidine salt. It is anticipated that a variety of chlorhexidine salts can be included in the antimicrobial polycarbonate coating such as, e.g., chlorhexidine acetate, diacetate, chloride, dichloride, hydrochloride, dihydrochloride, gluconate, or digluconate.

The antimicrobial polycarbonate may comprise one or more additional antimicrobial agent in addition to minocycline, rifampin, and chlorhexidine. For example, the antimicrobial polycarbonate coating may comprise gendine or gardine. Gendine is a combination of both chlorhexidine and the dye, Gentian violet. Gentian violet is a triarylmethane dye. Additionally, other dyes, such as Brilliant Green and food safe dyes such FD&C Blue No. 1 and FD&C Yellow No. 5. When Brilliant Green is combined with chlorhexidine, the combination is called Gardine. It is anticipated that these mixtures of dyes and chlorhexidine may improve antibiotic efficiency. A range of ratios of chlorhexidine to dye may be used, e.g., as described in U.S. Pat. No. 7,713,472.

A medium chain fatty acid or monoglyceride may be included in an antimicrobial polycarbonate coating as disclosed herein. The medium chain fatty acids or monoglyceride may have broad spectrum antimicrobial activity. Exemplary medium chain fatty acids that may be used include hexanoic, octanoic, decanoic and dodecanoic acids and their monoglycerides. The fatty acid may be a C₆₋₁₂ alkanoic acid or a C₆₋₁₀ alkanoic acid. The alkanoic acid may have 6, 7, 8, 9, 10, 11, or 12 carbons. In some embodiments, the fatty acid is octanoic acid. Without wishing to be bound by any theory, the medium chain fatty acid or monoglyceride may enhance membrane permeability or otherwise disrupt membrane function in a microorganism such as a bacteria. The medium chain fatty acid or monoglyceride may be combined with one or more antibiotics such as, e.g., minocycline and rifampin.

### D. Additional Therapeutic Agents

In some embodiments, the antimicrobial polycarbonate coating comprises an additional therapeutic agent. In embodiments where the antimicrobial polycarbonate coating comprises multiple layers, the additional therapeutic agent may be included in the same layer or in a different layer with the antimicrobial agents. For example, the additional therapeutic agent may be included in a layer of the antimicrobial polycarbonate coating as minocycline, rifampin, and/or chlorhexidine. While in some preferred embodiments the antimicrobial polycarbonate coating includes one or more antimicrobial agent and an additional therapeutic agent, it is nonetheless anticipated that the additional therapeutic agent may be included in a polycarbonate coating as described herein wherein the antimicrobial agents are not included in the polycarbonate coating. A variety of additional therapeutic agents may be included in the antimicrobial polycarbonate coating. In some embodiments, the additional therapeutic agent is an anticoagulant, a platelet inhibitor, a direct thrombin inhibitor, a calcium channel blocker, inhibitors of platelet or thrombin activation, thrombolytic agent, antibiotic, antifungal agent, antiseptics, antimicrobial metal, chelator (e.g., citrate, ethylene diaminedisuccinate (EDDS), or tetrakis hydroxymethyl phosphonium sulfate (THPF)), a fatty acid (e.g., a C₆₋₁₂ alkanoic acid, a C₆₋₁₀ alkanoic acid, hexanoic acid, decanoic acid, dodecanoic acid, caprylic acid (octanoic acid), caproic acid, or lauric acid), biofilm disrupter, vasodilator (e.g., glyceryl nitrate).

Anticoagulants, platelet inhibitors, and direct thrombin inhibitors can reduce vascular devices from becoming clogged or occluded. Thus, when included in a polycarbonate that is included on a vascular connector (e.g., a needleless connector) such compounds may reduce the probability of a downstream or connected catheter becoming clogged or occluded. The direct thrombin inhibitor may be lepirudin, desirudin, bivalirudin, or argatroban. Argatroban is an anticoagulant with the IUPAC systematic name of (2R,4R)-1-[(2S)-5-(diaminomethylideneamino)-2-┌┌(3R)-3-methyl-1,2,3,4-tetrahydroquinolin-8-yl┐sulfonylamino]pentanoyl]-4-methyl-piperidine-2-carboxylic acid]]. The platelet inhibitor may be, e.g., dipyridamole, ticagrelor, clopidogrel, or prasugrel. Dipyridamole (2,2′,2″,2′″-(4,8-di(piperidin-1-yl)pyrimido[5,4-d]pyrimidine-2,6-diyl)bis(azanetriyl)tetraethanol) can inhibit thrombus formation and promote vasodilation. The anticoagulant may be a glyceryl nitrate such as, e.g., glyceryl trinitrate (GTN). Glycerol nitrates can inhibit platelet activation (e.g., He'bert et al., 1997, Lacoste et al., 1994).

In some embodiments, the additional therapeutic agent is a calcium channel blocker. Calcium channel blockers may increase the supply of blood and oxygen to the heart. In some embodiments, the calcium channel blocker is included in a polycarbonate coating as described herein. The calcium channel blocker may be verapamil, amlodipine, nifedipine, diltiazem, thioridazine, or a thioridazine analogue. In some embodiments, the calcium channel blocker is a phenylalkylamine class L-type calcium channel blocker, such as, e.g., verapamil ((RS)-2-(3,4-dimethoxyphenyl)-5-{└2-(3,4-dimethoxyphenyl)ethyl┘-(methyl)amino}-2-prop-2-ylpentanenitrile]]) or thioridazine (10-{2-[(RS)-1-Methylpiperidin-2-yl]ethyl}-2-methylsulfanylphenothiazine]]). It is anticipated that a wide variety of therapeutic agents may be included in a polycarbonate coating or medical device (e.g., needleless connector for a catheter) of the present disclosure. In some embodiments, the calcium channel blocker is included in or coated on the polycarbonate coating.

### II. Methods of Producing Antimicrobial Polycarbonate Coatings

The antimicrobial polycarbonate coating may be applied to a surface via a variety of methods. For example, a polycarbonate may be dissolved in a solvent (e.g., dioxane or dioxalane) and then applied to a polymer surface, such as a polycarbonate surface of a medial device. A variety of concentrations of polycarbonate-solution may be used such as, e.g., about 1, 1.5, 2, 2.5, or 3% polycarbonate (w/v) in the solution (e.g., dioxane or dioxalane). The polycarbonate solvent solution can be applied to the surface of a medical device by dip-coating, painting, wiping, spray coating, or applying the solution onto the surface. After application, the coating can be dried (e.g., for several hours or one or more days at 37° C.).

The polycarbonate can be dissolved in a variety of solutions. The solution may be dioxane, dioxalane, tetrahydrofuran (THF), a chlorinated solvent, methylene chloride, chloroform, cresol, dimethylformamide, dimethylacetamide, of n-methyl pyrrolidone. In some embodiments, the solution is dioxane, as this solvent was observed to produce antimicrobial polycarbonate coatings with superior transparency as compared to dioxalane or THF. In some embodiments, the solvent is not THF; as shown in the below examples, use of THF as the solvent resulted in coatings that delaminated, tore, and did not display sufficient physical resiliency and toughness. Dioxane (1,4-dioxane) is a synthetic industrial chemical that is completely miscible in water (EPA 2006; ATSDR 2012). Synonyms for dioxane include dioxane, dioxan, p-dioxane, diethylene dioxide, diethylene oxide, diethylene ether, and glycol ethylene ether (EPA 2006; ATSDR 2012; Mohr 2001). In some embodiments, THF is not used as a solvent to dissolve the polycarbonate; nonetheless, THF can be included as a component of a mixed solvent system to produce a polycarbonate+carbothane blend (polycarbonate-urethane copolymer), wherein the polycarbonate can be dissolved in dioxane and the carbothane in THF, and then the different solutions are mixed to form the blended copolymer.

Antimicrobial agents may be dissolved in a solution comprising the polycarbonate prior to application to the surface of a medical device such as a polycarbonate surface. For example, one or more antimicrobial agents (e.g., minocycline, rifampin, and/or chlorhexidine diacetate) may be dissolved in a lower alcohol (e.g., a C₁₋₄ alcohol such as methanol) prior to mixing with the polycarbonate-solvent solution (e.g., the polycarbonate-dioxane solution). For example, an about 1%, 1.5%, 2%, 2.5%, 3%, or any range therein, polycarbonate in dioxane (w/v) solutions may be prepared. An about 0.1, 0.2, 0.3, 0.4, 0.5, 0.6, 0.7, 0.8, 0.9, 1, 1.1, 1.2, 1.25, 1.3, 1.4, 1.5, 1.75, 2.0% (w/v), or any range therein, of minocycline, rifampin and/or chlorhexidine diacetate in methanol solutions can be prepared (e.g., 0.6%, 1.2%, 1.25% w/v). The polycarbonate and antimicrobial solutions can be combined in a ratio of from about 99:1 to about 50:50, more preferably from about 95:5 to about 60:40, or about 55:45, 60:40, 65:35, 70:30, 75:25, or 80:20 ratio, or any range derivable therein. A medical device (e.g., a connector) can spray coated with the solution containing the polycarbonate and the antimicrobial agent(s). As shown in the below examples, it was observed that a polycarbonate-dioxane solution could be blended with a Minocycline or Rifampin or Chlorhexidine diacetate in methanol solution in a 70:30 ratio at 25° C. without precipitation.

The wt. ratio of polycarbonate to antimicrobial agent (w/w) in each layer may be about 2, 2.25, 2.4, 2.5, 3, 3.5, 3.9, 4, 4.5, 5, 6, 6.5, 7.1, 7.2, 7.3, 7.4, 7.5, 7.6, 7.7, 7.8, 7.9, 8, 8.25, 8.5, or any range derivable therein, (e.g., about 3.9 or 7.8). These methods can be used to produce antimicrobial polycarbonate coatings that are substantially transparent or translucent.

For example, the polycarbonate solution (e.g., polycarbonate in dioxane) may be about 7, 8, 9, 10, 11, 12, 13, 14, 15, 16, 17, 18, 19, 20 mg/mL, or any range derivable therein. The antimicrobial (minocycline, rifampin, and/or chlorhexidine) in methanol may be 10-40 mg/ml (e.g., 12, 24, or 36 mg/mL concentrations). CHX in methanol was 12 mg/mL (and 24 or 36 mg/mL in Example 7). In some embodiments, polycarbonate in dioxane (13 mg/mL) is mixed with minocycline and rifampin in methanol (12 mg/mL) at a 70/30 ratio (polycarbonate/antimicrobial) for coating; thus, the ratio antimicrobial to polycarbonate in the final (dried) coating layers were 0.4/1. The ratio of polycarbonate (e.g., polycarbonate or a polycarbonate blend) to antimicrobial agent may be from about 0.01/1 to about 0.7 in the dried polycarbonate coating, or 0.01, 0.1, 0.2, 0.3, 0.4, 0.5, 0.6, 0.7, or any range derivable therein.

As shown in the below examples, in some embodiments it has been surprisingly observed that antimicrobial polycarbonate coatings that contain lower concentrations of chlorhexidine can exert greater antimicrobial effects as compared to higher concentrations of chlorhexidine. Concentrations (2.4% and 3.6% w/v in methanol) used for chlorhexidine layers on different connectors (where 2.4% is denoted the 2× connector and 3.6% the 3× connector) were tested and it was observed that the 2× connector exhibited superior antimicrobial effects as compared to the 3× connector. The concentration of chlorhexidine in methanol may be, e.g., from about 2-3.5%, 2-3%, or about 2, 2.1, 2.2, 2.3, 2.4 2.4, 2.5, 2.6, 2.7, 2.8, 2.9, 3, 3.1, 3.2, 3.3, 3.4, or 3.5% w/v in methanol, or any range derivable therein. In some embodiments, the antimicrobial polycarbonate may be produced by mixing a solution containing about 1-36 mg/ml, more preferably about 10-24 mg/ml chlorhexidine diacetate in methanol and mixed with a polycarbonate-solvent (e.g., polycarbonate-dioxane) at a ratio of about 70:30 (w/v). The dried antimicrobial polycarbonate coating may contain about from 3% (w/w) (e.g., for a 1 mg/ml solution) to about 54% (w/w) (e.g., for a 36 mg/ml solution) chlorhexidine.

In some embodiments, the antimicrobial polymer coating comprises multiple layers. For example, the coating may include a polycarbonate layer containing minocycline layer (e.g., applied to a polycarbonate medical device such as a connector), a polycarbonate layer containing rifampin (e.g., applied after the minocycline layer), a polycarbonate layer containing chlorhexidine diacetate (e.g., applied after the rifampin layer).

In some embodiments, the ratio of minocycline:rifampin:chlorhexidine in an antimicrobial polycarbonate coating is about 1:1:1, 1:2:1, 2:1:1, 1:1:2, or any range derivable therein. Nonetheless, the different antimicrobial components may be applied in separate layers, optionally having different thicknesses, it is anticipated that a wide range of the possible ratios of antimicrobial agents can be used. For example, the difference in the ratios may be 100-fold different, e.g., if a very thin dilute layer of one is used and a thick concentrated layer of another is used). In some embodiments, the antimicrobial polycarbonate coating contains minocycline and rifampin, without chlorhexidine. In some embodiments, the (minocycline and rifampin) and chlorhexidine are included in separate layers in the antimicrobial polycarbonate coating; for example, the minocycline and rifampin may be included a first layer, and chlorhexidine can be separated into a second layer in the antimicrobial polycarbonate coating.

In some embodiments bioactive agents can be added as a suspension to the polymer coating solutions for application. As shown in the examples, in some instances including one or more bioactive agents in the coating solution nanoparticles are not included since translucency may be reduced in proportion to the quantity and size of insoluble present upon drying. It is anticipated that including nanoparticles (e.g., containing a bioactive agent, a therapeutic agent, or one or more antimicrobial agents) in the polycarbonate coating solution can allow for inclusion of the nanoparticles in the coating while minimizing or not significantly adversely reducing the translucency of the coating.

In some embodiments, nanoparticles are included in the antimicrobial polycarbonate coating. The nanoparticles may contain a bioactive agent, therapeutic agent, or antimicrobial agent (e.g., minocycline, rifampin, and/or chlorhexidine), as disclosed herein. Nanoparticles may minimize the reduction in optical properties while providing additional bioactive functionality. As shown in the below examples, the presence of nanoparticles did not impair the ability of the polycarbonate coating to strongly bond overlying laminate layers. In some embodiments, the nanoparticles include nanoparticles containing an antimicrobial agent, therapeutic compound, or drug as described herein or above. In some embodiments, the nanoparticles are gold nanoparticle conjugates. In some embodiments, the nanoparticles are a nanoemulsion or nanosuspension.

### III. Color-Changing Properties of Coatings

The antimicrobial polycarbonate coating may comprise one or more diagnostic indicators such as, e.g., a pH sensitive dye, indicator, or a marker specific to a disease state(s) or therapy. For example, pH sensitive dyes that may be used include litmus, phenolphthalein, and phenol red. The diagnostic indicator may include an antibody conjugate or other conjugate (e.g., comprising an enzyme or toxin) that can change color or produce a detectable signal when the target molecule binds. Conjugate dyes that can be used include, e.g., Texas Red, Rhodamine, Fluorescein, aminomethyl coumarin, phycoerythrin, cyanin derivates, alexa dyes, and thiol modified fluorescent dyes.

In some cases, it can be useful to include a colored molecule that can co-elute with one or more bioactive agents in the coating (e.g., an antimicrobial agent) over time. As the colored molecule slowly depletes from the antimicrobial polycarbonate coating, the color of the coating may decrease or become more clear. The change in color of the coating may signal to a clinician or patient that it may be time to replace a medical device, such as a needleless connector, comprising the antimicrobial polycarbonate coating.

In some embodiments, a color change may occur over time for antimicrobial polycarbonate coatings that contain minocycline and/or rifampin. A color change may occur from the slow elution of an antimicrobial agent or bioactive agent in the antimicrobial polycarbonate coating. For example, rifampin is a reddish color, and minocycline is yellow. For antimicrobial polycarbonate coatings that contain rifampin and/or minocycline, the slow elution of these compounds from a polycarbonate coating may result in a color change. This color change can be used as a visual cue that the antimicrobial protection has been reduced or depleted and it may be time to replace the medical device (e.g., connector) with a new one.

### IV. Medical Devices

The antimicrobial polycarbonate coatings can be applied to a variety of medical devices. The medical device may include materials that are stiff (e.g., having a high glass transition temperature), tough, and substantially transparent. In some embodiments, the medical device comprises polycarbonate-based polymers or polycarbonate polymer blends as described herein. The antimicrobial polycarbonate coating may be applied to a surface of medical devices including, e.g., connectors, clips, staples, housings, cartridges, trocars, tubes, barrels, plungers, and inflators. The antimicrobial polycarbonate coating may optionally contain one or more anticoagulants to reduce or prevent thrombus formation.

In some embodiments, the medical device is a vascular access device such as a needleless connector. Needleless connectors mediate flow between a source such as drip bag and an indwelling catheter to reduce or prevent hydraulic complications such as gas bubbles from forming in the lines. Needleless connectors may enable catheter access for infusion or aspiration. Needleless connectors were developed to reduce needlestick injuries by medical professionals. For prolonged intravenous therapies, these connectors are typically left in place for about 7 days. Transparency of the connectors can be important in visualizing any flow. These connectors are often fabricated from polycarbonate.

It is anticipated that the antimicrobial polycarbonate coatings can be applied to or included on a variety of needleless connectors. In some embodiments, an antimicrobial polycarbonate coating described herein is be applied to a needleless connector as described in U.S. Pat. Nos. 5,685,866, 5,873,862, 5,928,204, or U.S. Pat. No. 6,572,592. The connector can be, e.g., a connector or needleless connector as described in any one of U.S. Pat. Nos. 11,071,852, 10,799,692, 10,668,268, 10,391,293, 10,195,413, 9,775,981, 9,750,926, 9,440,060, 9,278,206, 8,758,306, or 6,682,509. Exemplary needleless connectors that may be coated with an antimicrobial polycarbonate coating as described herein include Clave® needleless connectors and MicroClave® connectors made by ICU Medical, Inc. Other needleless connectors that may be used with the present disclosure include CareSite®, SafeLine®, Invision Plus®, Ultrasite®, Nexus TKO®, MaxPlus, SmartSite™, MaxZero™, and One-Link needleless connectors.

In some embodiments, an interior surface of a needleless connector is coated with an antimicrobial polycarbonate coating as described herein. A portion of or all of a surface of the needleless connector may be coated with the antimicrobial polycarbonate coating. In some embodiments, both the internal surfaces facing the catheter and external surfaces facing away from the catheter of a needleless connector may be coated with an antimicrobial polycarbonate coating as described herein.

### IV. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Preparation of Polymer Coatings for Connectors

2% polycarbonate (w/v) was dissolved in Dioxane and Dioxalane. 7% Carbothane (polycarbonate urethane copolymer) (w/v) was dissolved in tetrahydrofuran (THF). Connectors were spray coated inside and outside allowing time to dry between spaying the top half and bottom half of the connector. Dioxane coating was transparent, Dioxalane and Carbothane were more cloudy. Connectors were connected and disconnected to catheter luers at the ends. Polycarbonate coatings withstood repeated cycles, Carbothane coating delaminated and tore. Connectors were also dipcoated with the same solutions yielding similar results.

### Example 2—Preparation of Polycarbonate Coatings Containing Antimicrobial Agents

2% polycarbonate in Dioxane solutions were prepared. 0.6% (w/v) Minocycline, Rifampin and Chlorhexidine diacetate in methanol solutions were prepared. Polycarbonate and antimicrobial solutions were combined in a 70:30 ratio. Connectors were spray coated with a Minocycline layer first, then a Rifampin layer and then a chlorhexidine diacetate layer. The wt. ratio of polycarbonate to antimicrobial agent (w/w) in each layer was 7.8. Clear coatings with a color tint (orange-brown) resulted. Different order of layering was also attempted with similar outcomes.

### Example 3: Preparation of Polycarbonate Coating with Different Polycarbonate-to-Antimicrobial Agent Ratios

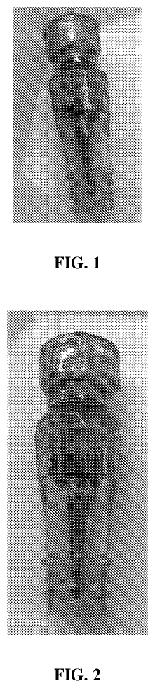

The connectors of Example 2 were prepared using 1.2% Minocycline, Rifampin and Chlorhexidine diacetate in methanol solutions. The wt. ratio of polycarbonate to antimicrobial agent (w/w) in each layer was 3.9. Resulting coated connectors were opaque. Another set of connectors were prepared using 1.25% Polycarbonate (w/v) in dioxane and 1.2% Minocycline, Rifampin and Chlorhexidine diacetate in methanol in a 70:30 ratio. Resulting coated connectors were transparent. The weight ratio of polycarbonate to antimicrobial agent in each layer was 2.4. Photograph of the triple layer coated connector is provided in FIG. 1.

It was possible to apply a topcoat of polycarbonate only by spray-coating on top of the laminated antimicrobial layers to potentially stabilize the antimicrobial agents and modulate elution kinetics. A photograph of the topcoated antimicrobial connector is provided in FIG. 2.

### Example 4: Microbiologic Testing

Microbial colonization challenges were conducted using clinical isolates of Staphylococcus aureus, Pseudomonas aeruginosa and Candida albicans. Connectors were immersed in plasma for 24 hrs and some in serum for an additional 6 days at 37 C. The different incubation durations will be referred to as 24 or 7 day challenges. Following incubation, microbial challenges were conducted. Inocula of 5 mL with 5.5×10⁵ CFU were added to a vial containing a conditioned connector and incubated at 37 C for 24 hr. Following 24 hr incubation with the inoculum, the connectors were rinsed with saline to remove non-adherent organisms then sonicated in DE neutralizing broth for 15 minutes to disrupt adherent biofilm. The sonicate solutions were then serially diluted and plated onto blood agar plates for quantitative enumeration. Viable colonies were counted following 24 hrs (48 hrs for yeast) incubation at 37 C. Results for 24 hr testing for Examples 2 and 3 are tabulated in Table 1X below.

TABLE 1X
Example 4

| PC (w/v) | M (w/v) | | CH (w/v) | | | | | | |
|---|---|---|---|---|---|---|---|---|---|
| % in | % in 30% | R (w/v) % | % in 30% | SA | | PA | PA | CA | CA |
| 70% of | of | in 30% of | of | control at | SA coated | control at | coated at | control at | coated at |
| coating | coating | coating | coating | 24 hrs | at 24 hrs | 24 hrs | 24 hrs | 24 hrs | 24 hrs |
| solution | solution | solution | solution | (CFU) | (CFU) | (CFU) | (CFU) | (CFU) | (CFU) |
| 2 | 0.6 | 0.6 | 0.6 | 1.2 × 10⁷ | 0 | 3 × 10⁶ | 1.1 × 10⁶ | 5.3 × 10⁶ | 4.1 × 10⁶ |
| 2 | 1.2 | 1.2 | 1.2 | 1.1 × 10⁸ | 0 | 5.2 × 10⁷ | 0 | 3.5 × 10⁶ | 5 × 10⁰ |
| 1.25 | 1.2 | 1.2 | 1.2 | 7.2 × 10⁶ | 0 | 1.8 × 10⁷ | 0 | 3.8 × 10⁶ | 1.7 × 10⁴ |

PC + M, PC + R and PC + CH layers coated sequentially
each layer's coating solution contained 70% PC in dioxane solution and 30% antimicrobial agent in methanol
PC = polycarbonate
M = minocycline
R = Rifampin
CH = chlorhexidine
SA = Staphylococcus aureus
PA = Pseudomonas aeruginosa
CA = Candida albicans

### Example 5: Polycarbonate—Chlorhexidine Antagonism Experiment

A silicone disk was spray coated and dried with a solution consisting of dioxane+polycarbonate mixed with chlorhexidine dissolved in methanol. A second disk was coated at the first except polycarbonate was omitted from the Dioxane solution. 24 hr microbial challenge was performed with Candida albicans using the same methodology as Example 4. The control disk had 9×10⁵ CFU, the polycarbonate containing coating had 1×10³ CFU and the non-polycarbonate chlorhexidine coating had 0 CFU. This indicates an antagonism between chlorhexidine and polycarbonate from the standpoint of antimicrobial effectiveness. While not being bound be any particular theory, Cattaneo et al report chlorhexidine can form relatively insoluble carbonate salts (Cattaneo et al, 2016).

### Example 6: Competitive Binding of Counterion with Polycarbonate to Improve Antimicrobial Availability

Control connectors were coated as in Example 3. A connector was coated where 2.4% Manganese acetate dissolved in methanol was added to the polycarbonate-dioxane solution prior to adding 2.4% chlorhexidine diacetate in methanol. The final dioxane/methanol ratio (v/v) was 70%/30% and the manganese diacetate/chlorhexidine diacetate ratio (w/w) was 1/1. Following drying, antimicrobial effectiveness against C. albicans was tested as in Example 4. The positive control had 2.8×10⁶ CFU and the manganese acetate had 5.5×10³ CFU. The log₁₀ reduction of the manganese acetate connector improved over the chlorhexidine without manganese acetate at 24 hr by 15%.

### Example 7: Higher Chlorhexidine to Polycarbonate Ratio

Connectors were coated as in Example 3 with 3 different chlorhexidine diacetate in methanol concentrations (2.4% and 3.6%) used for chlorhexidine layers on different connectors (where 2.4% is denoted the 2× connector and 3.6% the 3× connector). Microbiologic testing was performed as in Example 4. Results are tabulated below in Table 2×. The 2× connector surprisingly was more optimal in effectiveness than the 3× connector possibly because the relative percentage of CH to polymer became sufficiently high to enable breakthrough rapid elution.

TABLE 2X
Example 7

| PC (w/v) % | M (w/v) % | R (w/v) % | CH (w/v) % | | | | | | |
|---|---|---|---|---|---|---|---|---|---|
| in 70% of | in 30% of | in 30% of | in 30% of | SA control | SA coated | PA control | PA coated | CA control | CA coated |
| coating | coating | coating | coating | at 7 days | at 7 days | at 7 days | at 7 days | at 24 hrs | at 24 hrs |
| solution | solution | solution | solution | (CFU) | (CFU) | (CFU) | (CFU) | (CFU) | (CFU) |
| 1.25 | 1.2 | 1.2 | 2.4 | 1.3 × 10⁷ | 0 | 2.3 × 10⁷ | 0 | 6.2 × 10⁶ | 5 × 10⁰ |
| 1.25 | 1.2 | 1.2 | 3.6 | 1.3 × 10⁷ | 0 | 2.3 × 10⁷ | 455 | 6.2 × 10⁶ | 5 × 10⁰ |

PC + M, PC + R and PC + CH layers coated sequentially
each layer's coating solution contained 70% PC in dioxane solution and 30% antimicrobial agent in methanol
PC = polycarbonate
M = minocycline
R = Rifampin
CH = chlorhexidine
SA = Staphylococcus aureus
PA = Pseudomonas aeruginosa
CA = Candida albicans

### Example 8: Use of Polycarbonate—Polyurethane Blend Coating Layers

Connectors were coated as in Example 7 for the 2× chlorhexidine diacetate except the polycarbonate solution was replaced with a blend of polycarbonate and polycarbonate-urethane polymers. The blend consisted of a 50% polycarbonate in dioxane solution (1.25 w/v %) and 50% Carbothane (polycarbonateurethane copolymer) in tetrahydrofuran solvent (7 w/v %). Microbiologic testing was performed as described in Example 4. Results are tabulated below in Table 3X. Reducing the polycarbonate content by dilution with a polymer blend improved the antimicrobial durability.

TABLE 3X
Example 8

| PC (w/v) | PU (w/v) | M (w/v) | | | | | | |
|---|---|---|---|---|---|---|---|---|
| % in | % in 70% | % in 30% | R (w/v) % | CH (w/v) % | PA | PA | CA | CA |
| 70% of | of | of | in 30% of | in 30% of | control at | coated at | control at | coated at |
| coating | coating | coating | coating | coating | 7 days | 7 days | 24 hrs | 24 hrs |
| solution | solution | solution | solution | solution | (CFU) | (CFU) | (CFU) | (CFU) |
| 0.625 | 0.625 | 1.2 | 1.2 | 2.4 | 4.1 × 10⁷ | 0 | 1.0 × 10⁷ | 0 |

PC-PU + M, PC-PU + R and PC-PU + CH layers coated sequentially
each layer's coating solution contained 70% PC in dioxane solution and 30% antimicrobial agent in methanol
PC = polycarbonate
PU = polyurethane
M = minocycline
R = Rifampin
CH = chlorhexidine
PA = Pseudomonas aeruginosa
CA = Candida albicans

### Example 9_Additional Methods to Ensure Process Clarity of Polycarbonate Coatings

Samples were prepared as in Example 7 except Molecular Sieves were added to absorb any water from the environment that may have dissolved in the coating solution and reduced its clarity due to incompatible interaction with polycarbonate. 3 and 4 A size molecular sieves were added to the solutions prior to spray coating proceeded without issue. Coatings solutions could also be prepared in a dry nitrogen environment to further minimize hygroscopic artifacts.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 7,713,472
U.S. Pat. No. 10,195,413
U.S. Pat. No. 10,391,293
U.S. Pat. No. 10,668,268
U.S. Pat. No. 10,744,232
U.S. Pat. No. 10,799,692
U.S. Pat. No. 11,071,852
U.S. Pat. No. 5,685,866
U.S. Pat. No. 5,873,862
U.S. Pat. No. 5,928,204
U.S. Pat. No. 6,572,592
U.S. Pat. No. 6,682,509
U.S. Pat. No. 8,758,306
U.S. Pat. No. 9,278,206
U.S. Pat. No. 9,440,060
U.S. Pat. No. 9,750,926
U.S. Pat. No. 9,775,981
Cattaneo et al., Journal of molecular structure; 2016 Oct. 5; 1121:70-3, 2016.

What is claimed is:

1. A medical device comprising an antimicrobial polycarbonate coating, wherein the antimicrobial coating comprises an antimicrobial agent, and polycarbonate; wherein the antimicrobial agent is minocycline, rifampin, or chlorhexidine; and wherein the antimicrobial coating and has been formed by applying a solution comprising the antimicrobial agent and the polycarbonate to a surface of the medical device and allowing the solution to dry.

2. The medical device of claim 1, wherein the solution comprises dioxane, dioxalane, a chlorinated solvent, methylene chloride, chloroform, cresol, dimethylformamide, dimethylacetamide, or n-methyl pyrrolidone.

3. The medical device of claim 2, wherein the solution comprises dioxane.

4. The medical device of any one of claims 1-3, wherein the solution comprises a C₁₋₄ alcohol.

5. The medical device of claim 4, wherein the C₁₋₄ alcohol is methanol.

6. The medical device of any one of claims 1-5, wherein the polycarbonate comprises the structure 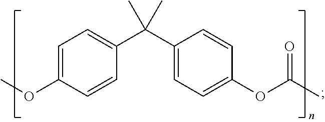
wherein n=4-18.

7. The medical device of any one of claims 1-6, wherein the polycarbonate comprises a clear polycarbonate, filled polycarbonate, polycarbonate blend, polycarbonate copolymer, polycarbonate block copolymer, high molecular weight polycarbonate, aliphatic polycarbonate, aromatic polycarbonate, or branched polycarbonate.

8. The medical device of any one of claims 1-6, wherein the polycarbonate comprises clear polycarbonate.

9. The medical device of any one of claims 1-8, wherein the ratio of polycarbonate to antimicrobial agent (w/w) is from about 2 to about 9.

10. The medical device of claim 9, wherein the ratio of polycarbonate to antimicrobial agent (w/w) is from about 2.25 to about 8.

11. The medical device of claim 9, wherein the ratio of polycarbonate to antimicrobial agent (w/w) is from about 2.25 to about 4.

12. The medical device of any one of claims 1-11, wherein the coating comprises a second polymer that is blended with the polycarbonate.

13. The medical device of claim 12, wherein the second polymer is a polyurethane, polypropylene, polyethylene, urethane, polyester, polyacrylate, acrylonitrile, rubber, polyalkenes, polysiloxane, polyethylene, polybutyl, polystyrene, polypropylene, polybutadiene, polysulfone, polyamide, or fluoropolymer.

14. The medical device of claim 13, wherein the second polymer is a polyurethane.

15. The medical device of claim 14, wherein the polyurethane comprises the structure 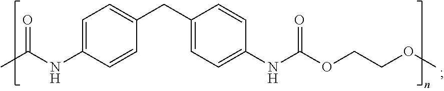
wherein n=4-18.

16. The medical device of any one of claims 12-15, wherein the ratio of polycarbonate to the second polymer is from about 99:1 (w/w) to about 50:50 (w/w).

17. The medical device of claim 16, wherein the ratio of polycarbonate to the second polymer is from about 95:5 (w/w) to about 65:45 (w/w).

18. The medical device of claim 16, wherein the ratio of polycarbonate to the second polymer is from about 90:10 (w/w) to about 75:25 (w/w).

19. The medical device of any one of claims 1-18, wherein the antimicrobial polycarbonate coating comprises (i) minocycline and rifampin, (ii) minocycline and chlorhexidine, or (iii) rifampin and chlorhexidine.

20. The medical device of claim 19, wherein the antimicrobial polycarbonate coating comprises minocycline and rifampin.

21. The medical device of claim 19, wherein the antimicrobial polycarbonate coating comprises minocycline, rifampin, and chlorhexidine.

22. The medical device of any one of claims 1-21, wherein the chlorhexidine is present in the coating in an amount of less than about 54% (w/w) when the coating is dried.

23. The medical device of claim 22, wherein the chlorhexidine is present in the coating in an amount of about 25-50% (w/w), or about 44% (w/w) or less, when the coating is dried.

24. The medical device of any one of claims 1-23, wherein the chlorhexidine is chlorhexidine diacetate, or chlorhexidine dichloride, chlorhexidine dihydrochloride, chlorhexidine digluconate, or a chlorhexidine base.

25. The medical device of any one of claims 1-24, wherein the minocycline, rifampin, and/or chlorhexidine are dissolved in a C₁₋₄ alcohol to form a first mixture, and the polycarbonate is dissolved or dispersed in dioxane, dioxalane, a chlorinated solvent, methylene chloride, chloroform, cresol, dimethylformamide, dimethylacetamide, or n-methyl pyrrolidone to form a second mixture, and wherein the first mixture and the second mixture are combined to form the solution comprising the antimicrobial agent and the polycarbonate.

26. The medical device of claim 25, wherein the C₁₋₄ alcohol is methanol.

27. The medical device of any one of claims 25-26, wherein the polycarbonate is dissolved or dispersed in dioxane.

28. The medical device of claim 27, wherein the ratio of polycarbonate to dioxane is from about 60:40 to about 80:20.

29. The medical device of claim 27, wherein the ratio of polycarbonate to dioxane is from about 65:35 to about 75:25.

30. The medical device of any one of claims 25-29, wherein the minocycline, rifampin, and/or chlorhexidine are dissolved in methanol in an amount of about 0.1-2% (w/v).

31. The medical device of claim 30, wherein the minocycline, rifampin, and/or chlorhexidine are dissolved in methanol in an amount of about 0.5-1.5% (w/v).

32. The medical device of any one of claims 30-31, wherein the minocycline, rifampin, and/or chlorhexidine are dissolved in methanol with no or essentially no precipitation of the minocycline, rifampin, and/or chlorhexidine.

33. The medical device of any one of claims 1-32, wherein the antimicrobial polycarbonate coating further comprises an additional antimicrobial agent.

34. The medical device of claim 33, wherein the additional antimicrobial agent is an antibiotic, an antifungal agent, an antiseptic, an antimicrobial metal, a biofilm disrupter, a chelator, or a fatty acid.

35. The medical device of claim 34, wherein the chelator is citrate, ethylene diaminedisuccinate (EDDS), tetrakis hydroxymethyl phosphonium sulfate (THPF), or EDTA.

36. The medical device of any one of claims 34-35, wherein the fatty acid is a C₆₋₁₂ alkanoic acid, more preferably a C₆₋₁₀ alkanoic acid.

37. The medical device of claim 36, wherein the fatty acid is hexanoic acid, decanoic acid, dodecanoic acid, caprylic acid (octanoic acid), caproic acid, or lauric acid.

38. The medical device of claim 37, wherein the fatty acid is caprylic acid (octanoic acid).

39. The medical device of any one of claims 34-38, wherein the antibiotic is a glycopeptide, a macrolide, a betalactam, a quinolone, a cephalasporin, a tetracycline, an aminoglycoside, a sulfa drug, an oxazolidinone, a nitrofuran, a betalactamase inhibitor, trimethoprim, tobramycin, clindamycin, doxycycline, tigecycline, or a combination thereof.

40. The medical device of any one of claims 34-39, wherein the antifungal agent is a polyene, an azole, an echinocandin, a mycovirus, or a mycophage.

41. The medical device of any one of claims 34-38, wherein the antiseptic is a peroxide, an antimicrobial metal compounds (e.g., silver, copper, zinc, titanium, barium, gold, gallium, nickel, aluminum, tellurium, selenium), a halogen (e.g., chlorine, iodine), sulfur, a sulfur compound, a phenolic, a quaternary ammonium compound, a halogenated phenol, an antimicrobial dye (e.g., methylene blue, brilliant green, crystal violet), an anatase titanium dioxide, a guanidium compound, a nitric oxide donor (e.g., SNAP and GSNO), a thionitrite, a NONOate, an antimicrobial peptide, an antimicrobial lipid (e.g., a fatty acid), a bacteriophage, a polymer comprising any of the foregoing, a nanoparticle comprising any of the foregoing, or a combination thereof.

42. The medical device of any one of claims 1-41, wherein the antimicrobial polycarbonate coating further comprises an additional therapeutic agent.

43. The medical device of claim 42, wherein the additional therapeutic agent is an anticoagulant, a platelet inhibitor, a direct thrombin inhibitor, a calcium channel blocker, inhibitors of platelet or thrombin activation, thrombolytic agent, or a vasodialator (e.g., glyceryl trinitrate).

44. The medical device of claim 43, wherein the additional therapeutic agent is an argatroban, a dabigatran, lepirudin, or bivalirudin.

45. The medical device of any one of claims 1-44, wherein the antimicrobial polycarbonate coating comprises nanoparticles.

46. The medical device of claim 45, wherein the nanoparticles comprise gold or a therapeutic agent.

47. The medical device of any one of claims 1-46, wherein the thickness of the antimicrobial polymeric coating is from about 1 micron to about 1 mm.

48. The medical device of any one of claims 1-47, wherein the applying comprises dip-coating, painting, wiping, or spray coating.

49. The medical device of any one of claims 1-46, wherein the antimicrobial polycarbonate coating is translucent or substantially transparent.

50. The medical device of claim 49, wherein the antimicrobial polycarbonate coating includes a visual cue to indicate that antimicrobial protection is depleting or wherein the color of the polycarbonate can change over time due to depletion of the antimicrobial agent from the antimicrobial polycarbonate coating.

51. The medical device of claim 49, wherein the medical device is a connector, and wherein the color of the polymeric coating can fade or become clearer due to elution of the minocycline and/or rifampin.

52. The medical device of any one of claims 1-51, wherein the medical device is a needleless connector, and wherein the needleless connector comprises a silicone seal and an internal cannula.

53. The medical device of claim 52, wherein the needleless connector comprises a split septum, a straight fluid path, and a clear or substantially transparent housing.

54. The medical device of any one of claims 1-53, wherein the surface of the medical device comprises polycarbonate, polyvinyl chloride (PVC), polypropylene (PP), polyethylene (PE), and polystyrene (PS), polyurethane, polysiloxane, fluoropolymer, polyester, or polyamide.

55. The medical device of any one of claims 1-54, wherein the surface of the medical device comprises or consists of polycarbonate.

56. The medical device of claim 55, wherein the medical device is a needleless connector, wherein the needleless connector comprises a polycarbonate housing.

57. The medical device of any one of claims 1-56, wherein the antimicrobial polycarbonate coating comprises multiple polycarbonate layers.

58. The medical device of claim 57, wherein the multiple layers comprise at least 1, 2, or 3 layers that comprise the minocycline, rifampin, and/or chlorhexidine.

59. The medical device of claim 58, wherein the antimicrobial polycarbonate coating comprises at least 2 layers that that comprise the minocycline, rifampin, and/or chlorhexidine that are separated by a polycarbonate layer that contains no or essentially no antimicrobial agent.

60. The medical device of claim 59, wherein the medical device comprises a first polycarbonate layer and a second polycarbonate layer; wherein the first polycarbonate layer comprises minocycline and rifampin, and wherein the second polycarbonate layer comprises chlorhexidine.

61. The medical device of claim 60, wherein the first polycarbonate layer and the second polycarbonate layer are separated by a third polycarbonate layer, wherein the third polycarbonate layer comprises no or essentially no antimicrobial agent.

62. The medical device of any one of claims 57-62, wherein the medical device comprises a polycarbonate layer comprises no or essentially no antimicrobial agent that is below the polycarbonate layers that contain the antimicrobial agent.

63. The medical device of claim 57, wherein the antimicrobial polycarbonate coating comprises a polycarbonate layer that comprises minocycline, rifampin, and chlorhexidine.

64. The medical device of any one of claims 1-63, wherein the antimicrobial polycarbonate coating comprises a polycarbonate topcoat, wherein the polycarbonate topcoat comprises no or essentially no antimicrobial agent.

65. The medical device of any one of claims 1-64, wherein the medical device is a connector, clip, staple, housing, cartridge, trocar, tube, barrel, plunger, or inflator.

66. The medical device of claim 65, wherein the medical device is a connector.

67. The medical device of claim 66, wherein the connector is a needleless connector.

68. A kit comprising the medical device of any one of claims 1-67 and a container means.

69. The kit of claim 68, wherein the medical device is a connector.

70. The kit of claim 69, wherein the connector is a needleless connector.

71. A method of reducing infection in a mammalian subject comprising using the medical device of any one of claims 1-64 in a medical procedure on the mammalian subject.

72. The method of claim 71, wherein the wherein the medical device is a connector, clip, staple, housing, cartridge, trocar, tube, barrel, plunger, or inflator.

73. The method of claim 72, wherein the medical device is a connector and wherein the medical procedure is intravenously administering a fluid to the mammalian subject via the connector.

74. The method of claim 73, wherein the connector comprises minocycline and/or rifampin, and wherein the becomes clearer or less colored over time as the fluid passes through the connector.

75. The method of claim 74, wherein the connector is a needleless connector.

76. A method of producing an antimicrobial polycarbonate coating, comprising:
(i) dissolving or dispersing an antimicrobial agent in a C₁₋₄ alcohol, to form a first solution;
(ii) dissolving or dispersing a polycarbonate in a solvent to form a second solution;
(iii) mixing the first solution and the second solution to form a third solution;
(iv) applying the third solution to a surface; and
(v) substantially drying the third solution to form the antimicrobial polycarbonate coating;
wherein the antimicrobial agent is minocycline, rifampin, or chlorhexidine.

77. The method of claim 76, wherein the surface is a on a medical device.

78. The method of any one of claims 76-77, wherein the surface is a polycarbonate surface on a medical device.

79. The method of any one of claims 76-78, wherein the applying comprises dip-coating, painting, wiping, or spray coating.

80. The method of any one of claims 76-79, wherein the drying occurs at room temperature.

81. The method of any one of claims 76-80, wherein the drying comprises the application of heated air or dry air.

82. The method of claim 76, wherein the solvent comprises dioxane, dioxalane, a chlorinated solvent, methylene chloride, chloroform, cresol, dimethylformamide, dimethylacetamide, or n-methyl pyrrolidone.

83. The method of claim 82, wherein the solvent comprises dioxane.

84. The method of any one of claims 76-83, wherein the C₁₋₄ alcohol is methanol.

85. The method of any one of claims 76-84, wherein the polycarbonate comprises the structure 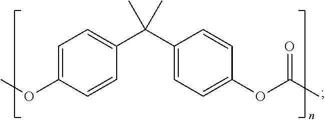
wherein n=4-18.

86. The method of any one of claims 76-85, wherein the polycarbonate comprises a urethane, polyester, polyacrylate, acrylonitrile, rubber, polyalkene, polysiloxane, polyethylene, polybutyl, polystyrene, polypropylene, polybutadiene, polysulfone, polyamide, or fluoropolymer.

87. The method of any one of claims 76-85, wherein the polycarbonate comprises clear polycarbonate.

88. The method of any one of claims 76-87, wherein the ratio of polycarbonate to antimicrobial agent (w/w) in the dried antimicrobial polycarbonate coating is from about 2 to about 9.

89. The method of claim 88, wherein the ratio of polycarbonate to antimicrobial agent (w/w) in the dried antimicrobial polycarbonate coating is from about 2.25 to about 8.

90. The method of claim 88, wherein the ratio of polycarbonate to antimicrobial agent (w/w) in the dried antimicrobial polycarbonate coating is from about 2.25 to about 4.

91. The method of any one of claims 76-90, wherein the C₁-4 alcohol is methanol; and wherein the solvent is dioxane.

92. The method of claim 91, wherein the ratio of polycarbonate to dioxane in the second solution is from about 60:40 to about 80:20.

93. The method of claim 92, wherein the ratio of polycarbonate to dioxane in the second solution is from about 65:35 to about 75:25.

94. The method of any one of claims 76-93, wherein the first solution comprises minocycline, rifampin, and/or chlorhexidine in methanol in an amount of about 0.1-2% (w/v).

95. The method of claim 94, wherein the first solution comprises minocycline, rifampin, and/or chlorhexidine in methanol in an amount of about 0.5-1.5% (w/v).

96. The method of any one of claims 76-95, wherein the first solution comprises minocycline, rifampin, and/or chlorhexidine dissolved in methanol with no or essentially no precipitation of the minocycline, rifampin, and/or chlorhexidine.

97. The method of any one of claims 76-96, wherein the second solution further comprises a second polymer that is blended with the polycarbonate.

98. The method of claim 97, wherein the second polymer is a polyurethane, polypropylene, polyethylene, urethane, polyester, polyacrylate, acrylonitrile, rubber, polyalkene, polysiloxane, polyethylene, polybutyl, polystyrene, polypropylene, polybutadiene, polysulfone, polyamide, or fluoropolymer.

99. The method of claim 98, wherein the second polymer is a polyurethane.

100. The method of claim 99, wherein the polyurethane comprises the structure 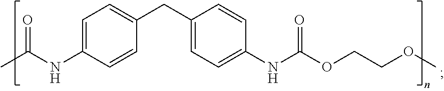
wherein n=4-18.

101. The method of any one of claims 97-100, wherein the ratio of polycarbonate to the second polymer in the dried antimicrobial polycarbonate coating is from about 99:1 (w/w) to about 50:50 (w/w).

102. The method of claim 101, wherein the ratio of polycarbonate to the second polymer in the dried antimicrobial polycarbonate coating is from about 95:5 (w/w) to about 65:45 (w/w).

103. The method of claim 101, wherein the ratio of polycarbonate to the second polymer in the dried antimicrobial polycarbonate coating is from about 90:10 (w/w) to about 75:25 (w/w).

104. The method of any one of claims 76-103, wherein the first solution comprises (i) minocycline and rifampin, (ii) minocycline and chlorhexidine, or (iii) rifampin and chlorhexidine.

105. The method of claim 104, wherein the first solution comprises minocycline and rifampin.

106. The method of claim 104, wherein the first solution comprises minocycline, rifampin, and chlorhexidine.

107. The method of any one of claims 76-106, wherein the first solution comprises chlorhexidine in methanol in an amount of less than about 3.6% (w/v).

108. The method of claim 107, wherein the first solution comprises chlorhexidine in methanol in an amount of about 2.4% (w/v) or less.

109. The method of any one of claims 76-108, wherein the chlorhexidine is chlorhexidine diacetate, or chlorhexidine digluconate, chlorhexidine dichloride, or chlorhexidine dihydrochloride.

110. The method of any one of claims 76-109, wherein the third solution further comprises an additional antimicrobial agent.

111. The method of claim 110, wherein the additional antimicrobial agent is an antibiotic, an antifungal agent, an antiseptic, an antimicrobial metal, a biofilm disrupter, a chelator, or a fatty acid.

112. The method of claim 111, wherein the chelator is citrate, ethylene diaminedisuccinate (EDDS), tetrakis hydroxymethyl phosphonium sulfate (THPF), or EDTA.

113. The method of any one of claims 111-112, wherein the fatty acid is a C₆₋₁₂ alkanoic acid, more preferably a C₆₋₁₀ alkanoic acid.

114. The method of claim 113, wherein the fatty acid is hexanoic acid, decanoic acid, dodecanoic acid, caprylic acid (octanoic acid), caproic acid, or lauric acid.

115. The method of claim 114, wherein the fatty acid is caprylic acid (octanoic acid).

116. The method of any one of claims 111-115, wherein the antibiotic is a glycopeptide, a macrolide, a betalactam, a quinolone, a cephalasporin, a tetracycline, an aminoglycoside, a sulfa drug, an oxazolidinone, a nitrofuran, a betalactamase inhibitor, trimethoprim, tobramycin, clindamycin, doxycycline, tigecycline, or a combination thereof.

117. The method of any one of claims 111-116, wherein the antifungal agent is a polyene, an azole, an echinocandin, a mycovirus, or a mycophage.

118. The method of any one of claims 111-115, wherein the antiseptic is a peroxide, an antimicrobial metal compounds (e.g., silver, copper, zinc, titanium, barium, gold, gallium, nickel, aluminum, tellurium, selenium), a halogens (e.g., chlorine, iodine), sulfur, a sulfur compound, a phenolic, a quaternary ammonium compound, a halogenated phenol, an antimicrobial dye (e.g., methylene blue, brilliant green, crystal violet), an anatase titanium dioxide, a guanidium compound, a nitric oxide donor (e.g., SNAP and GSNO), a thionitrite, a NONOate, an antimicrobial peptide, an antimicrobial lipid (e.g., a fatty acid), a bacteriophage, a polymer comprising any of the foregoing, a nanoparticle comprising any of the foregoing, or a combination thereof.

119. The method of any one of claims 76-118, wherein the third solution further comprises an additional therapeutic agent.

120. The method of claim 119, wherein the additional therapeutic agent is an anticoagulant, a platelet inhibitor, a direct thrombin inhibitor, a calcium channel blocker, inhibitors of platelet or thrombin activation, thrombolytic agent, or a vasodialator (e.g., glyceryl trinitrate).

121. The method of claim 120, wherein the additional therapeutic agent is an argatroban, a dabigatrans, lepirudin, or bivalirudin.

122. The method of any one of claims 76-121, wherein the third solution comprises nanoparticles.

123. The method of claim 122, wherein the nanoparticles comprise gold or a therapeutic agent.

124. The method of any one of claims 76-123, wherein the antimicrobial polycarbonate coating is translucent or substantially transparent.

125. The method of claim 124, wherein the antimicrobial polycarbonate coating comprises minocycline and/or rifampin, and wherein the color of the polymeric coating can fade or become clearer with use.

126. The method of claim 124, wherein the surface is on a medical device, wherein the medical device is a connector, and wherein the color of the polymeric coating can fade or become clearer due to elution of the minocycline and/or rifampin.

127. The method of any one of claims 76-125, wherein the surface is on a needleless connector, and wherein the needleless connector comprises a silicone seal and an internal cannula.

128. The method of claim 127, wherein the needleless connector comprises a split septum, a straight fluid path, and a clear or substantially transparent housing.

129. The method of any one of claims 76-128, wherein the surface comprises polycarbonate, polyvinyl chloride (PVC), polypropylene (PP), polyethylene (PE), and polystyrene (PS), polyurethane, polysiloxane, fluoropolymer, polyester, or polyamide.

130. The method of any one of claims 76-128, wherein the surface comprises or consists of polycarbonate.

131. The method of claim 130, wherein the medical device is a needleless connector, wherein the needleless connector comprises a polycarbonate housing.

132. The method of any one of claims 76-131, wherein the method comprises repeating steps (i)-(v), or wherein the method further comprises applying a fourth solution comprising the polycarbonate and the solvent to the surface.

133. The method of claim 132, wherein the method comprises repeating steps (i)-(v), to produce at least 1, 2, or 3 layers in the antimicrobial polycarbonate coating that comprise minocycline, rifampin, and/or chlorhexidine.

134. The method of claim 133, wherein the method comprises applying a fourth solution comprising the polycarbonate and the solvent to the surface, in order to produce at least two layers that that comprise the minocycline, rifampin, and/or chlorhexidine that are separated by a polycarbonate layer that contains no or essentially no antimicrobial agent.

135. The method of claim 134, wherein the dried antimicrobial polycarbonate coating comprises a first polycarbonate layer and a second polycarbonate layer; where the first polycarbonate layer comprises minocycline and rifampin, and wherein the second polycarbonate layer comprises chlorhexidine.

136. The method of claim 135, wherein the first polycarbonate layer and the second polycarbonate layer are separated by a third polycarbonate layer, wherein the third polycarbonate layer comprises no or essentially no antimicrobial agent.

137. The method of claim 132, wherein the dried antimicrobial polycarbonate coating comprises a polycarbonate layer that comprises minocycline, rifampin, and chlorhexidine.

138. The method of any one of claims 76-137, wherein the method further comprises applying a polycarbonate topcoat, wherein the polycarbonate topcoat comprises no or essentially no antimicrobial agent.

139. The method of any one of claims 76-138, wherein the method further comprises applying a polycarbonate basecoat, wherein the polycarbonate basecoat comprises no or essentially no antimicrobial agent.

140. The method of any one of claims 76-138, wherein the surface is on a medical device, wherein the medical device is a connector, clip, staple, housing, cartridge, trocar, tube, barrel, plunger, or inflator.

141. The method of claim 140, wherein the medical device is a connector.

142. The method of claim 141, wherein the connector is a needleless connector.
